(19) United States
(12) Patent Application Publication
(10) Pub. No.: US 2024/0274248 A1
(43) Pub. Date: Aug. 15, 2024

(54) METHOD AND SYSTEMS FOR MONITORING ELECTRONIC MEDICAL RECORD DATA ENTRY AND OVERFLOW

(71) Applicant: KONINKLIJKE PHILIPS N.V., EINDHOVEN (NL)

(72) Inventors: Francesco Sartor, Eindhoven (NL); Deep Bera, Bangalore (IN); Ron Martinus Laurentius VAN LIESHOUT, Geldrop (NL); Murtaza BULUT, Eindhoven (NL); Ralph Theodorus Hubertus MAESSEN, Eindhoven (NL)

(21) Appl. No.: 18/568,827

(22) PCT Filed: Jun. 9, 2022
(86) PCT No.: PCT/EP2022/065618

§ 371 Date: Dec. 10, 2023

Related U.S. Application Data
(60) Provisional application No. 63/210,153, filed on Jun. 14, 2021.

(51) Int. Cl. G16H 10/60; A61B 5/16

(52) U.S. Cl. CPC G16H 10/60; A61B 5/165; A61B 2503/24

(57) ABSTRACT

A method (100) for monitoring data entered into an electronic medical records (EMR) system (270), comprising: (i) estimating (104) an amount of information to be entered into the EMR system during a first monitoring period; (ii) determining (106) an amount of information entered into the EMR system during the first monitoring period; (iii) comparing (108) the determined amount of information entered into the EMR system during the first monitoring period to the estimated amount of information to be entered into the EMR system; (iv) estimating (110) a likelihood of error for information entered into the EMR system; and (v) assigning (112), based on the estimated likelihood of error, a reliability measure to the information entered into the EMR system or to information missing from the EMR system.

### FIELD OF THE DISCLOSURE

The present disclosure is directed generally to methods and systems for monitoring, using a data entry monitoring system, data entered into an electronic medical records (EMR) system.

### BACKGROUND

Electronic medical records (EMR) have provided a new level in the administration of patient information, and have thus increased the level of patient care. However, digitization of patient records also introduces significant new challenges and dangers.

One of the primary challenges in the digitization of patient records is the introduction of error. One of the many definitions of error is human entry error, such as by incorrectly entering data or omitting data. A primary cause of error is EMR interfaces. These interfaces are often not user friendly, and comprise problematic aspects such as hiding essential information where it is not directly reachable, graphical displays that obscure essential data, unwanted suggestions by the EMR software, and other aspects.

Another primary source of error is workflow. For instance, errors are more likely to occur when patients require more time than the time available for EMR bookkeeping. Indeed, clinicians must prioritize care of the patient and thus data entry into the EMR is necessarily a secondary activity, although a mandatory one. Often, the clinician's attention is not fully committed when entering the information in the EMR. Further, errors are more likely to occur in a situation of stress because of critical events. Moreover, workflow hurdles result in a backlog of information to be entered in the EMR, resulting in piled-up data. Missing information can be caused when dealing with patient's care and emergencies, such as instabilities, sudden changes, and more. This can be a vicious cycle, where incorrect or unreliable values are entered under a time pressure and thus the likelihood of errors increase. Errors can result in a lower level of care, thereby endangering the patient.

### SUMMARY OF THE DISCLOSURE

There is a continued need for methods and systems that monitor data entry into electronic medical record databases (EMR) and enable identification of problematic data.

Various embodiments and implementations herein are directed to a method and system configured to monitor data entered into an electronic medical records system. The method comprises: (i) estimating an amount of information to be entered into the EMR system during a first monitoring period; (ii) determining an amount of information entered into the EMR system during the first monitoring period; (iii) comparing the determined amount of information entered into the EMR system to the estimated amount of information to be entered into the EMR system; (iv) estimating, based on said comparing, a likelihood of error for the information entered into the EMR system during the first monitoring period; (v) assigning, based on the estimated likelihood of error, a reliability measure to the information entered into the EMR system or to information missing from the EMR system; and (vi) optionally labeling the information entered into the EMR system or the information missing from the EMR system based on the assigned reliability measure.

According to an aspect, a method for monitoring data entered into an electronic medical records (EMR) system is provided. The method includes: (i) estimating an amount of information to be entered into the EMR system during a first monitoring period, wherein the estimated amount of information to be entered into the EMR system further comprises an estimated time of entry of the information; (ii) determining an amount of information entered into the EMR system during the first monitoring period, wherein determining further comprises determining a time of entry of the information into the EMR system; (iii) comparing the determined amount of information entered into the EMR system during the first monitoring period to the estimated amount of information to be entered into the EMR system; (iv) estimating, based on said comparing, a likelihood of error for the information entered into the EMR system during the first monitoring period; and (v) assigning, based on the estimated likelihood of error, a reliability measure to the information entered into the EMR system or to information missing from the EMR system.

According to an embodiment, the method further includes the step of labeling the information entered into the EMR system or the information missing from the EMR system based on the assigned reliability measure.

According to an embodiment, a higher likelihood of error is assigned when: (i) information is entered into the EMR system after a lag period between obtaining the information and entering the information; (ii) the information is about a patient with a high risk healthcare status; (iii) the information is entered into the EMR system by an inexperienced healthcare professional; (iv) information is missing from the EMR system; (v) information is entered into the EMR system at a less reliable timepoint during a healthcare professional's shift; and/or (vi) information is entered into the EMR system using a mechanism with a higher error rate.

According to an embodiment, the estimated amount of information to be entered into the EMR system is based at least in part on one or more of: (i) a number of patients being monitored during the first monitoring period; (ii) a healthcare status of one or more of the patients being monitored during the first monitoring period; (iii) a number of healthcare professionals participating in monitoring during the first monitoring period; and/or (iv) an experience level of one or more healthcare professionals participating in monitoring during the first monitoring period; (v) availability of one or more of the healthcare professionals participating in monitoring during the first monitoring period; (vi) a timepoint during a healthcare professional's shift; (vii) a location of the healthcare professional during a shift; (viii) a mental or physical state of the healthcare professional; (ix) a monitoring situation of the patient; and (x) a guideline or protocol of a healthcare environment in which the patient is located.

According to an embodiment, the likelihood of error comprises one or more of no likelihood of error, a low likelihood of error, a medium likelihood of error, a high likelihood of error, and/or a value between 0 and 1.

According to an embodiment, the method further includes notifying a healthcare professional that information is missing from the EMR system.

According to an embodiment, the method further includes notifying a healthcare professional that information previously entered into or currently entered into the EMR system comprises a higher likelihood of error.

According to an embodiment, the healthcare professional is notified that information previously entered into or currently entered into the EMR system comprises a higher likelihood of error when the assigned reliability measure is below a predetermined reliability measure threshold.

According to an embodiment, labeling the information entered into the EMR system or the information missing from the EMR system with the assigned reliability measure comprises metadata.

According to an embodiment, the monitoring system comprises a patient monitor configured to provide information about one or more of: (i) a number of patients being monitored during the first monitoring period; (ii) a healthcare status of one or more of the patients being monitored during the first monitoring period; and (iii) an amount of information entered into the EMR system during the first monitoring period.

According to another aspect is a system configured to monitor electronic medical records (EMR) data entered into an EMR system. The system includes: a patient monitor configured to obtain information about a patient; a user interface configured to provide output to a healthcare professional and/or to receive input from the healthcare professional; and a processor configured to: (i) estimate an amount of information to be entered into the EMR system during a first monitoring period, wherein the estimated amount of information to be entered into the EMR system during the first monitoring period is based at least in part on the information about the patient obtained by the patient monitor; (ii) determine an amount of information entered into the EMR system during the first monitoring period, wherein determining further comprises determining a time of entry of the information into the EMR system; (iii) compare the determined amount of information entered into the EMR system during the first monitoring period to the estimated amount of information to be entered into the EMR system; (iv) estimate, based on said comparison, a likelihood of error for the information entered into the EMR system during the first monitoring period; and (v) assign, based on the estimated likelihood of error, a reliability measure to the information entered into the EMR system or to information missing from the EMR system.

According to an embodiment, the processor is further configured to notify, via the user interface, a healthcare professional that information previously entered into or currently entered into the EMR system comprises a higher likelihood of error, or that information is missing from the EMR system. According to an embodiment, the notification to the healthcare professional comprises a reason for the higher likelihood of error.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for monitoring data entered into an electronic medical records system, in accordance with an embodiment.

FIG. 2 is a schematic representation of a monitoring system, in accordance with an embodiment.

FIG. 3 is a graph depicting a relationship between data to be entered into an EMR system versus the time of entry of the data into an EMR system, in accordance with an embodiment.

FIG. 4 is a schematic representation of a monitoring system, in accordance with an embodiment.

FIG. 5 is a graph of data entry over time, in accordance with an embodiment.

### DETAILED DESCRIPTION OF EMBODIMENTS

The present disclosure describes various embodiments of a system and method configured to monitor data entry into electronic medical record databases (EMR). More generally, Applicant has recognized and appreciated that it would be beneficial to provide a method and system to enable the identification of problematic data entry into an EMR. The system includes a patient monitor configured to obtain information about a patient; a user interface configured to provide output to a healthcare professional; and a processor configured to: (i) estimate an amount of information to be entered into the EMR system, where the estimated amount of information to be entered into the EMR system during the first monitoring period is based at least in part on the information about the patient obtained by the patient monitor; (ii) determine an amount of information entered into the EMR system, wherein determining further comprises determining a time of entry of the information into the EMR system; (iii) compare the determined amount of information entered into the EMR system to the estimated amount of information to be entered into the EMR system; (iv) estimate, based on said comparison, a likelihood of error for the information entered into the EMR system during the first monitoring period; (v) assign, based on the estimated likelihood of error, a reliability measure to the information entered into the EMR system or to information missing from the EMR system; and (vi) label the information entered into the EMR system or the information missing from the EMR system with the assigned reliability measure. The system may be configured to notify, via the user interface, a healthcare professional that information entered into the EMR system comprises a higher likelihood of error, or that the information entered into the EMR system comprises a lower likelihood of error, or that information is missing from the EMR system.

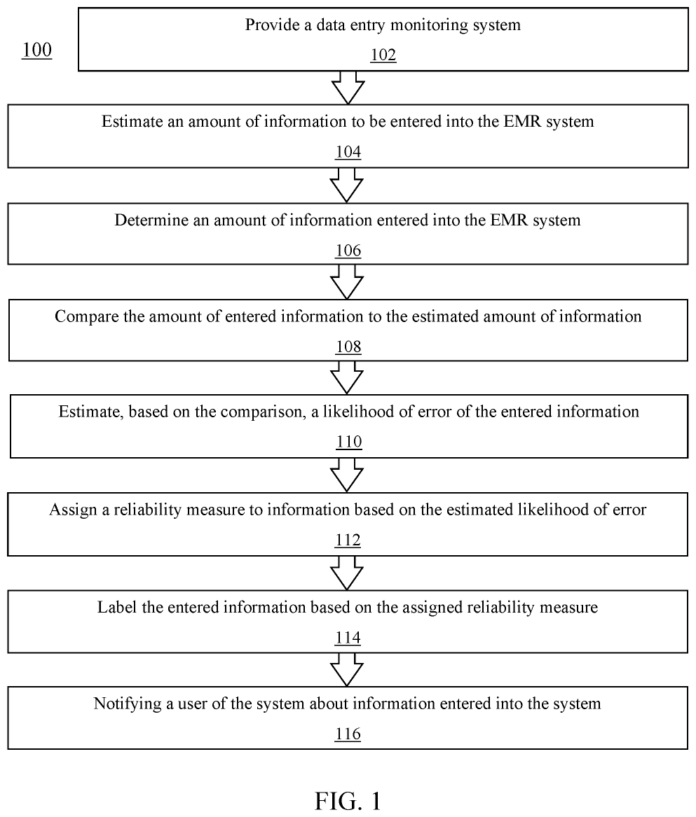

Referring to FIG. 1, in one embodiment, is a flowchart of a method 100 for monitoring, using a monitoring system, data entered into an electronic medical records system. The methods described in connection with the figures are provided as examples only, and shall be understood not to limit the scope of the disclosure. The monitoring system can be any of the systems described or otherwise envisioned herein. The monitoring system can be a single system or multiple different systems.

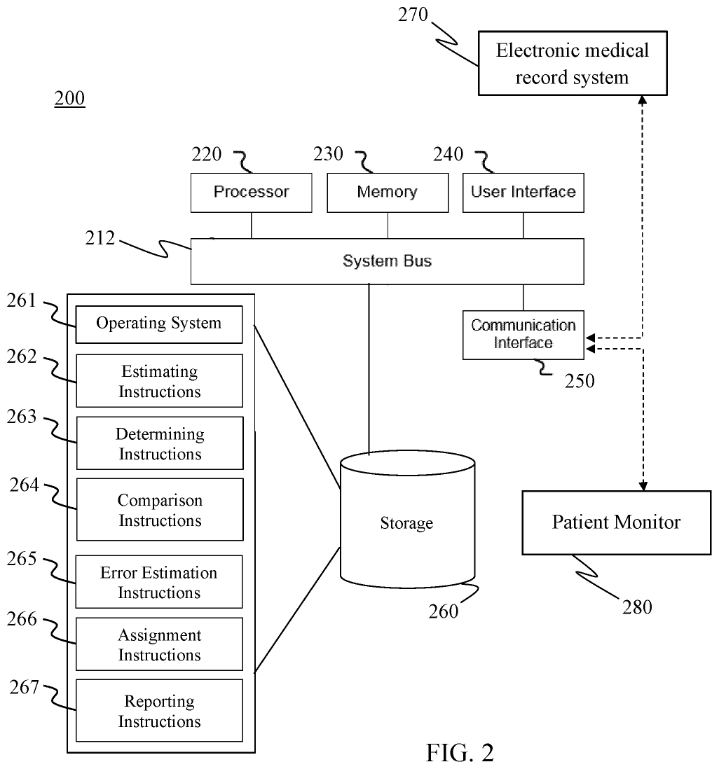

At step 102 of the method, a monitoring system 200 is provided. Referring to an embodiment of a monitoring system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, monitoring system 200 can be any of the systems described or otherwise envisioned herein. Other elements and components of the monitoring system 200 are disclosed and/or envisioned elsewhere herein.

According to an embodiment, the monitoring system comprises or is in direct or indirect communication with an electronic medical record system 270. The electronic medical record database or system comprises, among other possible data, one or more medical records for each of a plurality of patients currently undergoing care. The electronic medical record database or system can be any such database or system, including but not limited to the databases and systems described or otherwise envisioned herein.

According to an embodiment, the monitoring system comprises or is in direct or indirect communication with a patient monitor 280. The patient monitor can be any monitor, device, or system in communication with or otherwise receiving information from a patient or about a patient, including but not limited to the monitors and systems described or otherwise envisioned herein. According to an embodiment, the patient monitor is configured or designed to provide information about one or more of: (i) a number of patients being monitored during the first monitoring period; (ii) a healthcare status of one or more of the patients being monitored during the first monitoring period; and (iii) an amount of information entered into the EMR system during the first monitoring period, among many other points of information.

The monitoring system is an important tool that enables the identification and/or labeling of potentially problematic information or data entered into the electronic medical record system. The monitoring system can, for example, alert a clinician that data entered into the electronic medical record system is potentially problematic, which can improve patient care and outcomes. According to an embodiment, the monitoring system may comprise, be a component of, or be in communication with a clinical pathways management system that optimizes the treatment to patients.

According to an embodiment, the monitoring system is an integrated, multi-element or multi-component system that monitors or is otherwise aware of dataflow or data entry into an electronic medical records database or system. The monitoring system may be aware of or otherwise estimate or determine or know how much data, and/or what data, should be entered into the electronic medical records database or system with regard to a clinician, patient, and/or time period. The monitoring system may be aware of or otherwise estimate or determine or know how much data, and/or what data, is actually entered into the electronic medical records database or system with regard to that clinician, patient, and/or time period. Thus, the monitoring system can estimate, determine, or otherwise know or be aware of actual errors and/or likelihood of errors with regard to data entry into the electronic medical records database or system. According to just one possible embodiment among many others, the monitoring system therefore comprises information about: (i) a number of patients that it or a clinician, department, floor, or other grouping or cohort is monitoring, along with information about those patients such as demographics, critical condition levels, risk of instability, and more; (ii) a number of monitoring clinicians including their profiles, roles in treatment, short-term and/or long-term experience level (including number of hours on the ward, number of patients, patient types, crises dealt with, etc.), and other information, where a clinician can be any person interacting with or treating a patient, including but not limited to a physician, nurse, resident, technician, and more; (iii) patient care information, including but not limited to prognosis, treatment information, historical treatment information, historical health information, basic vital sign information such as ECG, blood pressure, SpO₂, etCO₂, and more, and/or advanced monitoring information such as echocardiogram, arterial blood pressure, pulmonary arterial pressure, and more.

At step 104 of the method, the system estimates an amount of information to be entered into the EMR system by a clinician during a first monitoring period. According to an embodiment, the estimated amount of information to be entered into the EMR system further comprises an estimated time of entry of the information. Notably, a clinician can be any person that enters information into an EMR system, including but not limited to a healthcare professional, technician, or any of a wide variety of other individuals.

The estimated amount of information to be entered into the EMR system by the clinician during the first monitoring period can be based on a wide variety of different factors, and can be determined by any of a variety of mechanisms utilized by the monitoring system. According to an embodiment, the amount of information to be entered into the EMR system by the clinician can be based at least in part on one or more of: (i) a number of patients being monitored during the first monitoring period; (ii) a healthcare status of one or more of the patients being monitored during the first monitoring period; (iii) a number of healthcare professionals participating in monitoring during the first monitoring period; and/or (iv) an experience level of one or more healthcare professionals participating in monitoring during the first monitoring period; (v) availability of one or more of the healthcare professionals participating in monitoring during the first monitoring period; (vi) a timepoint during a healthcare professional's shift; (vii) a location of the healthcare professional during a shift (where the location can be a past, current, and/or predicted future location); (viii) a mental or physical state of the healthcare professional (where the mental or physical state can be a past, current, and/or predicted future mental or physical state; (ix) a monitoring situation of the patient (wherein the amount of information can depend on the characteristics of the monitoring algorithms and monitoring devices (such as predictive algorithms based on machine learning, AI, digital twins, data analysis running in the sensors or devices, and so on); and (x) a guideline or protocol of a healthcare environment in which the patient is located (such as guidelines or protocols linked to EMR data acquisition, preparation, entry, and validation; these protocols can be specific to hospitals, and can have varying degrees of flexibility), among many other possibilities. For example, the timepoint during the healthcare professional shift (where a healthcare professional can be anyone caring for or otherwise responsible or in any way associated with a patient or care subject) could be the start of the professional's shift, during the professional's shift, at the end of the professional's shift, and/or a shift change, among other times.

While a healthcare professional can be anyone caring for or otherwise responsible or in any way associated with a patient or care subject, the healthcare professional can also be a person responsible for digitally storing the patient data into a data storage solution such as a database, or electronic medical records (EMR). Notably, the person collecting the data and person entering the data can be the same person, but they can also be different people. Further, the individuals may belong to different organizations, be in different locations, operate in different environments, be subject to different protocols and guidelines, have different performance metrics (KPIs), and so on. As just one example, the EMR information entry can be outsourced to a different organization, or there may be a situation where the information is entered via eICU, or tele-healthcare, or remote healthcare solutions. In these situations, calculating the error metric information about the both individuals and both organizations should be taken into account since the source of error can be due to the data collection, data storage, data access, data transfer, data interpretation, or data entry. In one embodiment, the method or system may even consider having separate error measures calculated for each of these aspects.

For example, with a larger number of patients monitored by a clinician or a department or floor or cohort, or a larger patient-to-clinician ratio, the amount of data to be entered into the EMR database or system may be greater and therefore there may be a greater likelihood of errors. Further, if one or more patients monitored by a clinician comprises a healthcare status that requires a higher level of monitoring, treatment, or other care, the amount of data to be entered into the EMR database or system may be greater and therefore there may be a greater likelihood of errors. Thus, if the monitoring system is aware of how many patients are being monitored by a clinician or other entity—such as by communicating with patient data and/or patient monitoring—then the monitoring system can estimate an amount of information that should or might be entered into the EMR database or system. This estimate can be determined based on a machine learning algorithm trained with historical data correlating the number of patients and/or their healthcare status with data entry, or can be based on other mechanisms.

As another example, with a smaller number of clinicians monitoring a group or other cohort of patients, the amount of data to be entered into the EMR database or system by any one clinician may be greater and therefore there may be a greater likelihood of errors. Further, the availability of the clinicians can affect the likelihood of errors during data entry. For example, if the clinician is monitoring patients while performing other duties, the clinician's attention may be less focused on data entry for the patients. Another potential effect on error entry is the experience level of the clinician caring for the patient and/or entering the information into the EMR database or system. As another example, timepoint during a healthcare professional's shift during which information is entered into the EMR database or system can affect the likelihood of error. The longer the clinician is into a shift, for example, or the more time-crunched a clinician may be, the greater the likelihood of error. Thus, if the monitoring system is aware of clinician staffing, roles, and/or experience level—such as by communicating with staffing and clinician demographic information—then the monitoring system can estimate an amount of information that should or might be entered into the EMR database or system. This estimate can be determined based on a machine learning algorithm trained with historical data correlating any or all of this clinician information with data entry, or can be based on other mechanisms. According to embodiment, therefore, the monitoring system may forecast the level of care required for the patients being monitored, and therefore can forecast the amount of data to be entered into the EMR database or system.

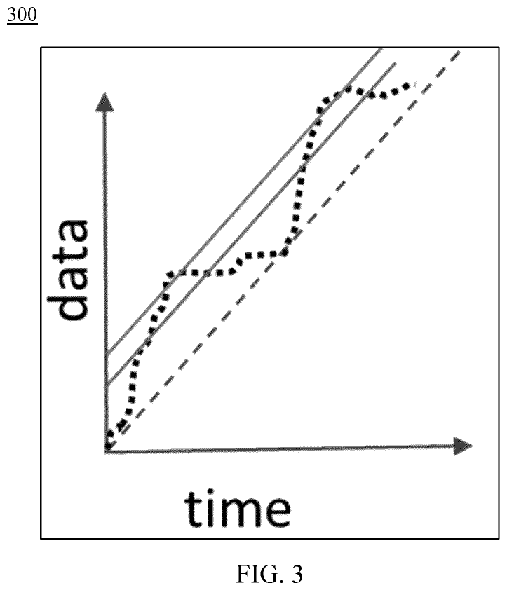

Referring to FIG. 3, in one embodiment, is a graph depicting a potential relationship between data to be entered into the EMR system (“data”) versus the time of entry of the data into the EMR system (“time”). When the expected amount of data is entered at an expected rate or within an expected time interval, the relationship is along or near the sloped dotted line (“expected”). The dotted line with changing slope (“actual”) is an example of the actual amount of data entered into the EMR system over time. When the actual amount of data entered into the EMR system over time is close to expected, actual is close to expected (where expected can also mean lower likelihood of error or no likelihood of error, among other thresholds or error levels). When the actual amount of data entered into the EMR system over time is more than expected, actual drifts toward up-left in the graph. This can indicate, for example, that there is a greater likelihood of error entry. For example, one threshold line may indicate a threshold for an intermediate risk of error entry, and another threshold line may indicate a high risk of error entry.

According to an embodiment, the monitoring system determines the data to be entered into the EMR database or system over time as the central dotted line. If staffing is on par with the data to be entered and validated, then the line showing the actual amount of data entered into the EMR system aligns with the central dotted line. When less data is entered and/or validated, the actual line will veer from the optimal central dotted line, thereby demonstrating a data entry lag (and thus demonstrating increased risk or likelihood of error).

At step 106 of the method, the system determines, measures, calculates, or otherwise identifies an amount of information entered into the EMR system during the first monitoring period. According to an embodiment, the system also determines a time of entry of the information into the EMR system. The monitoring system is in communication with the EMR database or system, or is a portal to the EMR database or system, or otherwise accesses or determines the amount of data entered by a clinician, potentially with information about the conditions under which the information was added, including but not limited to the time of entry, information about the clinician, and other contextual information.

According to an embodiment, the amount of information entered into the EMR system by the clinician during the first monitoring period can be determined by any of a variety of mechanisms utilized by the monitoring system. According to an embodiment, the amount of information entered into the EMR system by the clinician can be determined or measured directly by the monitoring system which is in direct or indirect communication with the EMR database or system.

According to an embodiment, information can be entered into a system using any of a variety of methods. For example, the information can be typed, such as manually inputting the information including but not limited to using a keyboard, speech-to-text based interfaces, gesture based interfaces, augmented, or virtual reality based interfaces, among other interfaces or input methods. Entering information can also mean verifying (i.e. checking, correcting if needed, and approving) information that has been created by an algorithm, or verifying (i.e. checking, correcting if needed, and approving) the information that has been created by another human, among others. According to an embodiment, the method of entry of information can contribute to the likelihood of error as described or otherwise envisioned herein.

At step 108 of the method, the system compares the determined amount of information entered into the EMR system to the estimated amount of information to be entered into the EMR system. According to an embodiment, the system compares the amount of information entered into the EMR system during the first monitoring period to the estimated amount of information to be entered into the EMR system during the first monitoring period, and/or compares the amount of information entered into the EMR system by a particular clinician and/or about a particular patient to the estimated amount of information to be entered into the EMR system by the particular clinician and/or the particular patient. According to an embodiment, the comparison can be made by any of a variety of mechanisms utilized by the monitoring system.

According to an embodiment, the comparison can be made as information is entered into the EMR database or system, or can be compared after the information is entered into the EMR database or system. For example, the comparison may comprise a comparison of stored data regarding what information was entered into the EMR system at what time. The results of the comparison can be utilized immediately or stored in local or remote storage for use in further steps of the method.

At step 110 of the method, the system estimates, based on the comparison in step 108 of the method, a likelihood of error for the information entered into the EMR system during the first monitoring period. According to an embodiment, the estimate can be determined using any of a variety of mechanisms utilized by the monitoring system.

According to an embodiment, a higher likelihood of error might be estimated or assigned when: (i) information is entered into the EMR system after a lag period between obtaining the information and entering the information; (ii) the information is about a patient with a high risk healthcare status; (iii) the information is entered into the EMR system by an inexperienced healthcare professional; (iv) information is missing from the EMR system; and/or (v) information is entered into the EMR system at a less reliable timepoint during a healthcare professional's shift.

The determined or estimated likelihood of error can be qualified or quantified in a wide variety of different ways. For example, the likelihood of error can comprise one or more of no likelihood of error, a low likelihood of error, a medium likelihood of error, a high likelihood of error, and/or a value between 0 and 1, among many other classifications or labels.

According to an embodiment, the method of entry of information can contribute to the likelihood of error. For example, information entered via keyboard or via speech recognition (speech-to-text) may inherently comprise a higher likelihood of error, while information entered via an algorithm or other mechanism may inherently comprise a lower likelihood of error (or vice versa).

According to an embodiment, the estimated likelihood of error can be utilized immediately or stored in local or remote storage for use in further steps of the method.

Notably, the system can similarly estimate or assign a lower likelihood that the information entered into the EMR system comprises an error, and/or can notify a user when the likelihood of error is below a certain risk or error threshold.

According to an embodiment, the system re-compares at step 108 and/or re-estimates at step 110, a likelihood of error for information at a subsequent time point. For example, step 108 and/or 110 can comprise both initial information entered into the system and subject to comparison and estimate, and can further comprise subsequent information entered into the system. Thus, the initial information at time T may comprise a likelihood estimate, and this estimate can influence—or not influence—the comparison and/or estimate for information at time T+t. Many other variations are possible.

According to an embodiment, the system can similarly estimate or assign a likelihood that information not yet entered into the system comprises a lower or higher likelihood of error. For example, the system can determine that expected information is missing from the system, and can assign or determine or estimate a likelihood of error for that information. For example, the system may assign or determine or estimate a likelihood of error for that information based on similar or associated or historical information entered into the system. For example, the system may determine that individual X is known to have a high error rate, and thus the information that should have been entered by individual X but was not entered, is likely to have a high likelihood of error (or vice versa, if individual X is known to have a low error rate, the information that should have been entered by individual X but was not entered is likely to have a low likelihood of error). Many other methods of assigning or determining or estimating a likelihood of error for missing information are possible.

According to an embodiment, the system may assign or determine or estimate a likelihood of error for information entered into or missing from the system based on human input. For example, a user may examine the information entered into or missing from the system and determine a likelihood of error. The likelihood of error may be selected from a determined list of options, the user may provide input given a calculated error, may enter text in an open form, or may otherwise assign or determine or estimate a likelihood of error for information.

At step 112 of the method, the system assigns a reliability measure to information entered into the EMR system or to information missing from the EMR system, based on the estimated likelihood of error. According to an embodiment, the reliability measure can be determined using any of a variety of mechanisms utilized by the monitoring system. The reliability measure may be a weight, a label, or any of a wide variety of other measures. According to an embodiment, labeling the information entered into the EMR system or the information missing from the EMR system with the assigned reliability measure comprises metadata associated with information entered into or missing from the EMR database or system.

Notably, according to an embodiment, the reliability measure is simply the likelihood of error calculated or estimated in step 110 of the method. Additional or alternatively, the reliability measure is any modification, adjustment, weight, or other aspect of the likelihood of error calculated or estimated in step 110 of the method.

According to an embodiment, therefore, the monitoring system updates and/or accounts for a risk or stability status based on information entered into the EMR database or system. In the event information was entered under circumstances that may indicate a high likelihood of error, or information that was expected to be entered but was not entered, the monitoring system assigns a reliability measure to the entered or missing information. The system may also utilize information about clinician staffing, experience, and/or workload, among many other factors. The information can be utilized by the monitoring system, for example, to compute the EMR data entry requirements versus time availability of staff. Additionally, a historical record of successful entries of staff may be accounted for by the monitoring system when assigning risk and/or a reliability measure. The monitoring system computes and updates the patient acuity level, the instability risk, clinician experience, and/or clinician work-pressure, among other factors. These derived data are then used to estimate a likelihood of error. Missing—but expected—data can be treated in this context as error. The monitoring system can know that the expected data is missing as it can obtain this information from the EMR database or system. According to an embodiment, the monitoring system provides a safety net when evaluating EMR data by enabling the medical staff either directly involved in the data entered and validated or consulting such data later in time, that such data have been assigned a low, moderate, or high reliability measure, among other possible reliability weight measure types or parameters. According to an embodiment, the monitoring system can further qualify the reliability measure by providing a root cause analysis of events that led to the particular reliability measure assignment made by the monitoring system.

According to an embodiment, the assigned reliability measure can be utilized immediately or stored in local or remote storage for use in further steps of the method.

According to an embodiment, the system may assign the likelihood of error based on human input. For example, a user may examine the information entered into or missing from the system and determine a likelihood of error and/or the reliability measure (which could optionally be just the likelihood of error, among many other measures). The likelihood of error may be selected from a determined list of options, the user may provide input given a calculated error, may enter text in an open form, or may otherwise assign or determine or estimate a likelihood of error for information, and this may be used to assign the reliability measure.

At step 114 of the method, the system labels the information entered into the EMR system or the information missing from the EMR system based on the assigned reliability measure. According to an embodiment, the monitoring system can label information entered into the EMR system, or information missing from the EMR system, with the assigned reliability measure using any of a variety of mechanisms. For example, the information entered into or missing from the EMR database or system can be directly labeled with the assigned reliability measure, such as via labeling the information or missing information as metadata or any other labeling. As another example, the information entered into or missing from the EMR database or system can be associated in memory with the assigned reliability measure. As another example, the information entered into or missing from the EMR database or system can be associated with the assigned reliability measure in a lookup table or other reference mechanism.

According to an embodiment, the system may label the information with the likelihood of error based on human input. For example, a user may examine the information entered into or missing from the system and determine a likelihood of error and/or the reliability measure (which could optionally be just the likelihood of error, among many other measures), and thus direct the system to label the information with that reliability measure.

According to an embodiment, the information labeling can be utilized immediately or stored in local or remote storage for use in further steps of the method.

At step 116 of the method, the system optionally notifies or alerts a clinician or other individual that information is missing from the EMR system, and/or that information previously entered into or currently entered into the EMR system comprises a higher likelihood of error. According to an embodiment, the monitoring system can generate a notification or alert using any of a variety of different mechanisms. According to one embodiment, the clinician or other individual is notified that information entered into the EMR database or system comprises a higher likelihood of error when the assigned reliability measure is above a predetermined reliability measure threshold, and/or when information is missing from the EMR database or system. According to another embodiment, the system optionally notifies or alerts a clinician or other individual that information is entered into the system, and/or that the information previously entered into or currently entered into the EMR system comprises any likelihood of error, including but not limited to no likelihood of error, a low likelihood of error, or any other level of likelihood of error.

According to an embodiment, the notification or alert may be provided to a user via a user interface of the monitoring system. Thus, the alert may be text, sound, haptic feedback, a visualization, text, email, or any other type of notification to a user. The notification or alert may comprise information about the entered or missing information, the time period, the patient(s), the clinician(s), and any other information relevant to a user's analysis and/or understanding of the notification or alert. According to an embodiment, the monitoring system may provide, via a user interface, a visualization of the notification or alert. The visualization can be any visualization that enables a user's analysis and/or understanding of the relevant information.

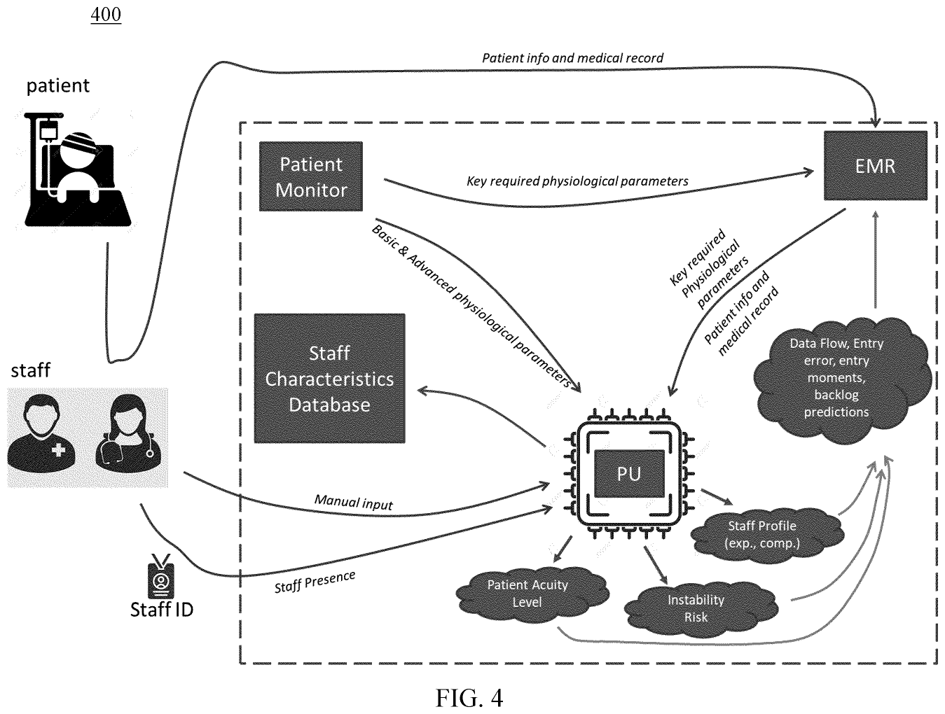

Referring to FIG. 4, in one embodiment, is a schematic representation of a monitoring system 400. The system is in communication with or monitoring one or more activities or other information about one or more patients (such as via “Patient Monitor”) and one or more clinicians (“staff”). Information about the staff such as staffing, experience, and workload can be received by the monitoring system (“Staff Characteristics Database”) as well. The monitoring system comprises an EMR database or system (“EMR”) into which information is entered from a variety of sources such as the patient monitor, a clinician, and other sources of information. The monitoring system monitors data flow into the EMR database or system, compares that to expected information to be entered into the EMR database or system, and determines a risk of error. The monitoring system can be in communication with a wide variety of information sources, including but not limited to a pharmacy information system, a laboratory information system, a radiology information system, a clinical data repository (CDR), nursing documentation (DOC), electronic medication administration record (eMAR), clinical decision support system (CDS), a computerized physician order entry system (CPOE), and many other systems.

### EXAMPLE

The following example utilizing a monitoring system is only one possible embodiment and/or example. Accordingly, it is understood that it is a non-limiting example of the monitoring system.

In this example, a clinician entered a lab parameter, namely lactate, measured from patient A into the EMR record file of patient B. This is a data entry error caused by the clinician. The lactate lab parameter is in a physio-pathological range, so the monitoring system does not know this is an error by itself The monitoring system can calculate the likelihood that this data was entered incorrectly, and know the circumstances under which it was entered, including the time that it was entered. For example, perhaps the lactate date was entered when the data stack was lagging behind a particular threshold, such as 30 values per minute per staff member. This is just one example of a particular threshold, and many variations are possible. Moreover, perhaps the lactate data was entered after a period of emergency, by a staff member with three months activity on the ward. As a result, the lactate data is ranked with a low reliability measure due to data stack overview, junior clinician/user, and post-crisis entry.

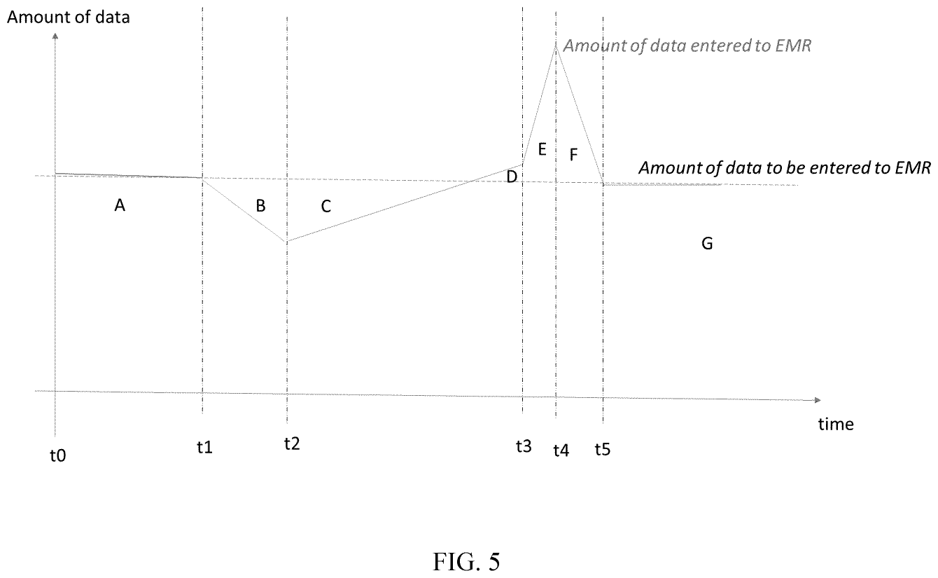

Referring to FIG. 5, in one embodiment, is a graph of data versus time, with a charted line of the amount of data entered into the EMR database or system over time as well as a charted line of the amount of data expected to be entered into the EMR database or system over time. Although the line demonstrating the amount of data expected to be entered into the EMR database or system over time is relatively stable in FIG. 5, it should be appreciated that this amount of data can vary widely over time. In this example, period A (shown by “A” in the graph) is a period in which demand (i.e., the data expected to be entered into the EMR database or system) equals the supply (i.e., the data actually entered into the EMR database or system). In period B, the demand exceeds the supply, and there is a negative slope as supply is decreasing. In period C, the demand still exceeds the supply, but there is a positive slope as supply is increasing. In period D, supply is exceeding demand. The slope is positive as the clinician is catching up with data entry. In period E, supply is greater than demand with a larger positive scope, and the clinician is still catching up with data entry. In period F, supply still exceeds demand, but the slope is negative as data entry is getting caught up and/or slowing. In period G, demand is approximately equal to supply. According to an embodiment, the system may determine, for example, that periods B and C are at least equal to periods D, E, and F to ensure that all required data is entered.

According to an embodiment, during time periods corresponding to regions B and C less than required baseline data is entered, while during time periods D, E, and F more data than the baseline is entered to compensate for the periods B and C. Since more than is entered during D, E, F, assuming that the resources for data entry (i.e., number of people, and time spent) has not changed, the likelihood or error during these periods is greater than other periods (e.g., A, B, C, and G). Further, one can compare the areas under curve divided by time to calculate the amount of data entered per unit time for regions D, E and F to determine for which one of these the likelihood of error will be greater. In one embodiment, when the rate of information entry is greater than the baseline or than the value established in protocols, then the likelihood of error is positively correlated with the rate of information being entered.

According to an embodiment, the amount of data expected to be entered into the EMR database or system over time can be calculated or estimated using a wide variety of mechanisms. According to one embodiment, a standard operating procedure (SOP) or other guidelines may be utilized. For example, the SOP/guidelines may indicate that data is required to make an assessment or decision, and thus the demand value is increased. According to one embodiment, an algorithm may be utilized such that if the prediction power of the algorithm goes down the demand value for the data goes up. The system may also indicate which data type is in higher demand. According to one embodiment, the clinical situation of the patient and/or other values may indicate a deterioration suggesting an increased demand value. According to one embodiment, handover of patient increases the likelihood of errors and the need for more data to better judge the error rate.

According to an embodiment, an error likelihood threshold can be calculated in a wide variety of ways. The simplest implementation is an empirical approach, such as based on literature. Another approach is data driven, where the monitoring system calculates one or more Key Performance Indicators relevant for that specific ward (e.g., rate of re-hospitalization, rate of mortality, rate of complications, average length of staying, rate of beds occupancy, and/or many others) and relates this in general linear model of with machine learning algorithm with the level of data stack overflow. Another way is by labeling errors that have been discovered by medical staff itself. A hybrid model of those outlined here is possible as well. According to an embodiment, the monitoring system can keep in memory the unfilled data and remind to the staff in a moment when given the acuity of patients and the staff availability this task can be carried out.

According to an embodiment, a data driven implementation can be utilized to calculate the reliability measure, where a machine learning algorithm can be trained to calculate reliability weights, or to select functions that can calculate reliability weights, or to calculate outputs which can be used by a (mathematical) function or another machine learning algorithm to calculate reliability weights. Many different types of input data can be used for training. Further, different categories of data can be considered for calculating input features, such as information about data, information about user and workflow, and information about EMR settings.

According to an embodiment, the monitoring system can consider data characteristics. Examples of data characteristics include a timestamp. Different timestamps can be considered, such as: (i) timestamps as defined by the protocols (the suggested best time to enter information into EMR), (ii) time of the actual data collection, and (iii) time of the entry of the data into EMR. The origin/source of data can be considered, such as: monitor data (e.g., patient vitals), device data (e.g., ventilator data, ultrasound data), human input (e.g., user notes), lab data, medication data, financials, and more. According to an embodiment, the system can consider the source of the data when it is data generated by a predictive or descriptive algorithm, such as software to calculate patient stability, digital twins running simulation to evaluate patient state, ultrasound image analysis algorithms, MRI segmentation algorithms, and many other types of software or algorithms. The type of data can be considered, such as: text, numbers, symbols, images, video, speech, and more. Also considered can be the amount of data, amount of data entered per a given time, data entry rate, and various other measures and statistics derived from the amount of data, and duration of the data entry (such as max rate of date entry, average rate of data entry, etc.). The creator of data can be considered, such as: human (via notes and annotations), sensors, algorithms, and more. The medium of creation of the original data can be considered, such as: paper notes (which are then translated to EMR), electronic notes (notes created on computer), automatic (data created by sensors/monitors, algorithms), and more. The method of creating the EMR data can be considered, such as: via typing the data, via copy & paste, via speech-to-text, auto completion by EMR, via automatic data creation by EMR followed by user verification, and more. Characteristics of the record creator can be considered, such as: is the person entering the data in EMR the same as the person that created (e.g. measured, noted, processed) the data, and more. Whether the data is generated by an algorithm (machine learning) can be considered. The amount of data in the source (e.g. in the notes) vs. amount of data entered in EMR can be considered by the monitoring system. Whether the format of data in the source is the same as the format of the data entered (e.g., data in source is bulleted while the EMR data is detailed explanations; monitor data is waveforms while EMR entries are statistics or parts of these waveforms; medication database includes full drug names while EMR data comprises abbreviations, and more). The completeness/fragmentation of data can be considered, such as: whether data corresponding to the current entry is complete or fragmented, whether the complete data is entered all at once or at different time points, whether the complete data is entered by the same person, and more. The heterogeneity/homogeneity of data can be considered, such as: whether data is coming from single source/type (e.g., only monitor data, or monitor data plus nurse notes, and more. The type of data entered can be considered, such as: namely whether the type and amount of the data entered to EMR has previously been entered by the same person.

In addition, user and workflow characteristics can be considered by the monitoring system. For example, the following characteristics can be considered: user clinical experience, user experience with EMR, number and type of user interruptions during the data entry, workflow related information (how busy is the unit, at which stage of the shift is data entered such as the beginning, middle, or end of shift), what were the significant events in the corresponding unit (such as crisis, patient deterioration, new problems, rounds, etc.) before or during the data entry, and/or personal aspects such as how was the clinical feeling (e.g., tired, stressed, emotional, and more).

In addition, EMR and context-related features can be considered by the monitoring system. For example, the following characteristics can be considered: the device, software, or tool used to enter data in EMR; the location of the user when the data was entered; the setting(s) of the EMR during the data entry (which features are activated, error check, auto completion, verification using algorithm, and more); display crowdedness, how many windows open, other data visualized, number of clicks, and more; and duration of data entry, among many other aspects.

With these and/or other considerations, the monitoring system can be trained to calculate reliability measures. For training of such an algorithm, a labeled data set comprising errors for existing EMR entries, or comprising labeled/annotated missing EMR entries can be used.

According to an embodiment, the monitoring system utilizes patient condition information, or learning from population data, or from similar patients, to predict the amount of data that should have been entered in a certain time period. Comparing the amount of the entered information to the information that should have been entered, as a preventative measure, means that alerts can be generated to target nurses or other users of the EMR database or system. For instance, the user can be alerted about how delaying the entry to the data will influence the reliability of data, such as: “if you delay data entry by X amount of time, the risk of errors or missing data will increase by Y%”. The current patient state can be used to quantify the effects of delaying the data entry into the EMR database or system.

Referring to FIG. 2 is a schematic representation of an integration system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, processor 220 may comprise, among other instructions or data, estimating instructions 262, determining instructions 263, comparison instructions 264, error estimation instructions 265, assignment instructions 266, and/or reporting instructions 267.

According to an embodiment, estimating instructions 262 direct the system to estimate an amount of information to be entered into the EMR system by a clinician during a first monitoring period. According to an embodiment, the estimated amount of information to be entered into the EMR system further comprises an estimated time of entry of the information. Notably, a clinician can be any person that enters information into an EMR system, including but not limited to a healthcare professional, technician, or any of a wide variety of other individuals. The estimated amount of information to be entered into the EMR system by the clinician during the first monitoring period can be based on a wide variety of different factors, and can be determined by any of a variety of mechanisms utilized by the monitoring system.

According to an embodiment, determining instructions 263 direct the system to determine, measure, calculate, or otherwise identify an amount of information entered into the EMR system during the first monitoring period. According to an embodiment, the system also determines a time of entry of the information into the EMR system. The monitoring system is in communication with the EMR database or system, or is a portal to the EMR database or system, or otherwise accesses or determines the amount of data entered by a clinician, potentially with information about the conditions under which the information was added, including but not limited to the time of entry, information about the clinician, and other contextual information. According to an embodiment, the amount of information entered into the EMR system by the clinician during the first monitoring period can be determined by any of a variety of mechanisms utilized by the monitoring system. According to an embodiment, the amount of information entered into the EMR system by the clinician can be determined or measured directly by the monitoring system which is in direct or indirect communication with the EMR database or system.

According to an embodiment, comparison instructions 264 direct the system to compare the determined amount of information entered into the EMR system to the estimated amount of information to be entered into the EMR system. According to an embodiment, the system compares the amount of information entered into the EMR system during the first monitoring period to the estimated amount of information to be entered into the EMR system during the first monitoring period, and/or compares the amount of information entered into the EMR system by a particular clinician and/or about a particular patient to the estimated amount of information to be entered into the EMR system by the particular clinician and/or the particular patient. According to an embodiment, the comparison can be made by any of a variety of mechanisms utilized by the monitoring system. According to an embodiment, the comparison can be made as information is entered into the EMR database or system, or can be compared after the information is entered into the EMR database or system.

According to an embodiment, error estimation instructions 265 direct the system to estimate a likelihood of error for the information entered into the EMR system during the first monitoring period. According to an embodiment, the estimate can be determined using any of a variety of mechanisms utilized by the monitoring system.

According to an embodiment, assignment instructions 266 direct the system to assign a reliability measure to information entered into the EMR system or to information missing from the EMR system, based on the estimated likelihood of error. According to an embodiment, the reliability measure can be determined using any of a variety of mechanisms utilized by the monitoring system. The reliability measure may be a weight, a label, or any of a wide variety of other measures. According to an embodiment, labeling the information entered into the EMR system or the information missing from the EMR system with the assigned reliability measure comprises metadata associated with information entered into or missing from the EMR database or system.

According to an embodiment, reporting instructions 267 direct the system to notify or alert a clinician or other user that information is missing from the EMR system, and/or that information previously entered into or currently entered into the EMR system comprises a higher likelihood of error. According to an embodiment, the monitoring system can generate a notification or alert using any of a variety of different mechanisms. According to one embodiment, the clinician or other individual is notified that information entered into the EMR database or system comprises a higher likelihood of error when the assigned reliability measure is above a predetermined reliability measure threshold, and/or when information is missing from the EMR database or system. According to an embodiment, the monitoring system 200 notifies or alerts the user via a user interface 240 of the system, and/or via other mechanisms. The system may alert the user via any mechanism for alert, including but not limited to a visual display, an audible notification, a text message, an email, a page, or any other method of notification.

It is important to note that the method for monitoring, using a monitoring system, can be utilized for a wide variety of data systems other than for data entered into an electronic medical records system. Accordingly, where the present specification utilizes “electronic medical records” and/or “electronic medical records system,” the terms can be replaced with “data” or “data records” or [insert data type] records” for “electronic medical records,” and the corresponding data system. Accordingly, the system and method could be can be applied anywhere there is a digital record maintained by specialized staff and critical operations which impair the regular inflow of data entry in such system. For instance, the methods and systems could work as well in a OR ward or other similar wards within a hospital setting. However, the methods and systems could just as easily work in a non-hospital setting.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for monitoring, using a monitoring system, data entered into an electronic medical records (EMR) system, comprising:
estimating an amount of information to be entered into the EMR system during a first monitoring period, wherein the estimated amount of information to be entered into the EMR system further comprises an estimated time of entry of the information;
determining an amount of information entered into the EMR system during the first monitoring period, wherein determining further comprises determining a time of entry of the information into the EMR system;
comparing the determined amount of information entered into the EMR system during the first monitoring period to the estimated amount of information to be entered into the EMR system;
estimating, based on said comparing, a likelihood of error for the information entered into the EMR system during the first monitoring period; and
assigning, based on the estimated likelihood of error, a reliability measure to the information entered into the EMR system or to information missing from the EMR system.

2. The method of claim 1, further comprising the step of labeling the information entered into the EMR system or the information missing from the EMR system based on the assigned reliability measure.

3. The method of claim 1, wherein a higher likelihood of error is assigned when one or more of:
(i) information is entered into the EMR system after a lag period between obtaining the information and entering the information;
(ii) the information is about a patient with a high risk healthcare status; (iii) the information is entered into the EMR system by an inexperienced healthcare professional;
(iv) information is missing from the EMR system;
(v) information is entered into the EMR system at a less reliable timepoint during a healthcare professional's shift; or
(vi) information is entered into the EMR system using a mechanism with a higher error rate.

4. The method of claim 1, wherein the estimated amount of information to be entered into the EMR system is based at least in part on one or more of:
(i) a number of patients being monitored during the first monitoring period;
(ii) a healthcare status of one or more of the patients being monitored during the first monitoring period;
(iii) a number of healthcare professionals participating in monitoring during the first monitoring period or (iv) an experience level of one or more healthcare professionals participating in monitoring during the first monitoring period;
(v) availability of one or more of the healthcare professionals participating in monitoring during the first monitoring period;
(vi) a timepoint during a healthcare professional's shift; (vii) a location of the healthcare professional during a shift;
(viii) a mental or physical state of the healthcare professional; (ix) a monitoring situation of the patient; or
(x) a guideline or protocol of a healthcare environment in which the patient is located.

5. The method of claim 1, further comprising the step of notifying a healthcare professional that information is missing from the EMR system.

6. The method of claim 1, further comprising the step of notifying a healthcare professional that information previously entered into or currently entered into the EMR system comprises a higher likelihood of error.

7. The method of claim 1, wherein the healthcare professional is notified that information previously entered into or currently entered into the EMR system comprises a higher likelihood of error when the assigned reliability measure is below a predetermined reliability measure threshold.

8. The method of claim 2, wherein labeling the information entered into the EMR system or the information missing from the EMR system with the assigned reliability measure comprises metadata.

9. The method of claim 1, wherein the monitoring system comprises a patient monitor configured to provide information about one or more of:
(i) a number of patients being monitored during the first monitoring period;
(ii) a healthcare status of one or more of the patients being monitored during the first monitoring period; or
(iii) an amount of information entered into the EMR system during the first monitoring period.

10. A system configured to monitor electronic medical records (EMR) data entered into an EMR system, comprising:
a patient monitor configured to obtain information about a patient;
a user interface configured to provide output to a healthcare professional and/or to receive input from the healthcare professional; and
a processor configured to:
(i) estimate an amount of information to be entered into the EMR system during a first monitoring period, wherein the estimated amount of information to be entered into the EMR system during the first monitoring period is based at least in part on the information about the patient obtained by the patient monitor;
(ii) determine an amount of information entered into the EMR system during the first monitoring period, wherein determining further comprises determining a time of entry of the information into the EMR system;
(iii) compare the determined amount of information entered into the EMR system during the first monitoring period to the estimated amount of information to be entered into the EMR system;
(iv) estimate, based on said comparison, a likelihood of error for the information entered into the EMR system during the first monitoring period; or
(v) assign, based on the estimated likelihood of error, a reliability measure to the information entered into the EMR system or to information missing from the EMR system.

11. The system of claim 10, wherein the estimated amount of information to be entered into the EMR system is based at least in part on one or more of:
(i) a number of patients being monitored during the first monitoring period;
(ii) a healthcare status of one or more of the patients being monitored during the first monitoring period;
(iii) a number of healthcare professionals participating in monitoring during the first monitoring period or an experience level of one or more healthcare professionals participating in monitoring during the first monitoring period;
(vi) availability of one or more of the healthcare professionals participating in monitoring during the first monitoring period;
(vii) a timepoint during a healthcare professional's shift;
(viii) a location of the healthcare professional during a shift;
(ix) a mental or physical state of the healthcare professional; a monitoring situation of the patient; or
(x) a guideline or protocol of a healthcare environment in which the patient is located, wherein the estimated amount of information to be entered into the EMR system further comprises an estimated time of entry of the information.

12. The system of claim 10, wherein determining an amount of information entered into the EMR system during the first monitoring period comprises determining a time of entry of the information into the EMR system.

13. The system of claim 10, wherein a higher likelihood of error is assigned when one or more of:
(i) information is entered into the EMR system after a lag period between obtaining the information and entering the information;
(ii) the information is about a patient with a high risk healthcare status;
(iii) the information is entered into the EMR system by an inexperienced healthcare professional;
(iv) information is missing from the EMR system;
(v) information is entered into the EMR system at a less reliable timepoint during a healthcare professional's shift; or
(vi) information is entered into the EMR system using a mechanism with a higher error rate.

14. The system of claim 10, wherein the processor is further configured to notify, via the user interface, a healthcare professional that information previously entered into or currently entered into the EMR system comprises a higher likelihood of error, or that information is missing from the EMR system.

15. The system of claim 14, wherein the notification to the healthcare professional comprises a reason for the higher likelihood of error.
